(19) United States
(12) Patent Application Publication
(10) Pub. No.: US 2005/0203330 A1
(43) Pub. Date: Sep. 15, 2005

(54) Disposable saline water cartridge module for radiopharmaceuticals dispensing and injection system

(76) Inventors: Akio Muto, Shonandai Fujisawa-City (JP); Chi-Min Hu, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/044,196

(22) Filed: Jan. 28, 2005

(30) Foreign Application Priority Data
Feb. 6, 2004 (TW) 93201681

(51) Int. Cl. A61M 36/14; A61K 51/00

(52) U.S. Cl. 600/1

(57) ABSTRACT

A saline water cartridge for radiopharmaceuticals injection includes a casing in which a primary passage extends from an outlet of a saline water reservoir to a radiopharmaceuticals inlet port of the cartridge and a secondary passage extends from an opening defined in the primary passage to a radiopharmaceuticals outlet port where a patient injection device is connected to supply the radiopharmaceuticals to the patient. A first one-way membrane valve is arranged in the opening of the primary passage to allow the radiopharmaceuticals injected into the primary passage from a radiopharmaceuticals carrying syringe to flow to the outlet port, while preventing reverse flow. A second one-way membrane valve is arranged in the outlet of the saline water reservoir to allow saline water to replenish into the primary passage while preventing reverse flow.

### FIELD OF THE INVENTION

The present invention relates generally to a saline water cartridge, and in particular to a saline water cartridge for radiopharmaceuticals injection.

### BACKGROUND OF THE INVENTION

Positron emission tomography (PET) has been recently used in invivo imaging, which helps early detection of cancers for early treatment. This makes PET one of most important measures for diagnosis of a variety of cancers. Positron radionuclides for PET treatment are generated in a cylcotron and then composed with other elements to form compound/molecules, such as glucose, amino acid, and water for injection into human body. The positrons are annihilated with electrons inside the human body, which emits gamma ray that can be detected by PET equipment for imaging.

The PET facility is of great help for medical diagnosis, but the positron radionuclides of the PET radiopharmaceuticals give off strong radiation, which, if not properly shielded, may cause serious damage to the medical employees, who are in charge of handling, dispensing, transportation, and injection of the radiopharmaceuticals for PET. For example, to minimize the radiation residual in a syringe that carries the pharmaceutical medicine, the syringe barrel must be repeatedly flushed with saline water and the saline water be injected into the body of the patient. Such a repeated operation often makes the finger or other portion of the body of the PET operator over-exposed to the radiation of the radiopharmaceuticals.

Conventional arrangements for radiation shielding often emphasize in providing a casing or container to prevent leakage of radiation of the radiopharmaceuticals received or accommodated therein. However, in handling the radiopharmaceuticals, the medical employees, such as technicians and nurses, still face the risk of radiation damage during retrieval, disposition, dispensing, quality inspection, and injection of the radiopharmaceuticals.

Thus, it is desired to have a nuclear radiation safe system to handle radiopharmaceuticals and in particular to have a saline water cartridge that cooperates with such a system to minimize the nuclear radiation hazard for medical employees.

### SUMMARY OF THE INVENTION

A primary objective of the present invention is to provide a saline water cartridge that cooperates with a radiopharmaceuticals dispensing and injection system to perform injection of the radiopharmaceuticals with the minimized hazard of nuclear radiation damage.

Another objective of the present invention is to provide a saline water cartridge for a radiopharmaceuticals dispensing and injection system, comprising a casing inside which internal passages are formed for connection among a radiopharmaceuticals inlet port, a radiopharmaceuticals output port, and an outlet of saline water, which ensures proper and smooth of the radiopharmaceuticals from the inlet port to the outlet port, together with saline water supplied from a saline water reservoir arranged inside the cartridge.

A further objective of the present invention is to provide a saline water cartridge for radiopharmaceuticals injection, which cartridge can be disposed of after to the injection in order to eliminate undesired exposure and contamination of radiation.

Yet a further objective of the present invention is to provide a saline water cartridge featuring easy operation and nuclear radiation safety; which eliminates manual operation of repeatedly flushing the radiopharmaceuticals carrying syringe with saline water and thus reduces nuclear radiation hazard.

To achieve the above objects, in accordance with the present invention, there is provided a saline water cartridge for radiopharmaceuticals injection, comprising a casing in which a primary passage extends from an outlet of a saline water reservoir to a radiopharmaceuticals inlet port of the cartridge and a secondary passage extends from an opening defined in the primary passage to a radiopharmaceuticals outlet port where a patient injection device is connected to supply the radiopharmaceuticals to the patient. A first one-way membrane valve is arranged in the opening of the primary passage to allow the radiopharmaceuticals injected into the primary passage from a radiopharmaceuticals carrying syringe to flow to the outlet port, while preventing reverse flow. A second one-way membrane valve is arranged in the outlet of the saline water reservoir to allow saline water to replenish into the primary passage while preventing reverse flow.

The saline water cartridge in accordance with the present invention, when incorporated in a radiopharmaceuticals dispensing and injection system effectively enhances the operation efficiency of the radiopharmaceuticals dispensing and injection system and also helps increasing radiation safety for radiopharmaceuticals injection. Further, the saline water cartridge of the present invention helps automatizing the radiopharmaceuticals dispensing and injection system. Manual operation of repeatedly flushing the syringe that carries the radiopharmaceuticals can be replaced by automatic process, which enhances radiation safety.

### BRIEF DESCRIPTION OF THE DRAWINGS

The present invention will be apparent to those skilled in the art by reading the following description of a preferred embodiment thereof, with reference to the attached drawings, in which:

FIG. 1 is a perspective view illustrating a radiopharmaceuticals dispensing and injection system in which a disposable saline water cartridge constructed in accordance with the present invention is incorporated;

FIG. 2 is a perspective view of a portion of the radiopharmaceuticals dispensing and injection system, particularly showing a carrier of a dispensing and injection mechanism moving a syringe that carries liquid radiopharmaceuticals to an injection position for injection of the liquid into the disposable saline water cartridge of the present invention for further injection into a patient, a syringe plunger being in an injected condition;

FIG. 3 is similar to FIG. 2, but showing the syringe plunger in a withdrawn condition;

FIG. 4 is a cross-sectional view showing the syringe that carries the liquid radiopharmaceuticals penetrating into the disposable saline water cartridge with the plunger in the injected condition;

FIG. 5 is similar to FIG. 4, but showing the plunger in the withdrawn condition; and

FIG. 6 is a perspective view of the disposable saline water cartridge constructed in accordance with the present invention.

### DETAILED DESCRIPTION OF PREFERRED EMBODIMENTS

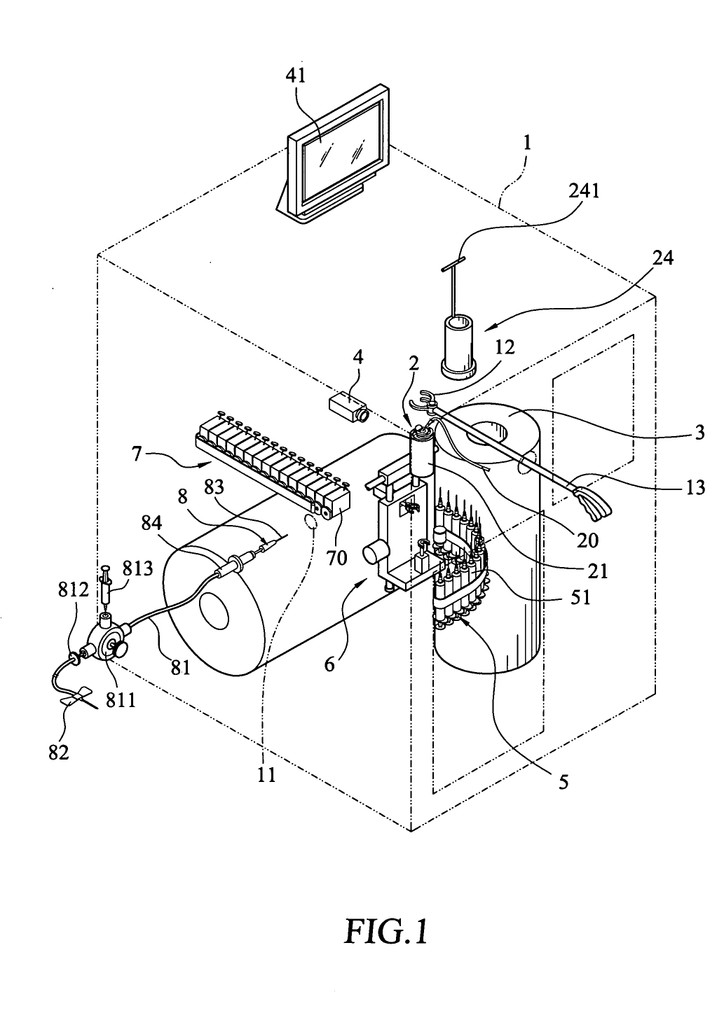

With reference to the drawings and in particular to FIG. 1, a radiopharmaceuticals dispensing and injection system (which will be abbreviated as “the system” hereinafter) is shown. The system comprises a sealed casing 1, which defines a radiation-shielded chamber delimited by side walls and top and bottom walls (all not labeled). A hole 11, serving to receive an injection device 8 for supply a liquid radiopharmaceuticals to a patient, is defined in one side wall of the casing 1. A vial 2, which contains radiopharmaceuticals in liquid form or liquid-carrying form, is arranged inside the radiation-shielded chamber of the casing 1.

The system comprises a manipulating arm 12 supported and operated by a handle 13 extending out of the casing 1 for manual operation or coupled to other automatic driving system (not shown). The manipulating arm 12 selectively and releasably grasps the vial 2 for positioning the vial 2 in a vial container 21, where a radiopharmaceuticals supply tube 20 is removably connected to the vial 2 for supplying a predetermined amount of radiopharmaceuticals to the vial 2. Monitoring of the supplying of the radiopharmaceuticals to vial 2 is performed by means of for example a monitoring device 4, such as a charge-coupled device (CCD) based camera, which takes images of the vial 2 via an observation window (not shown) formed in a side wall of the vial container 21. The images are transmitted and displayed on a monitor display 41 or a computer device for visual inspection or further processing. Level of the medicine filled into the vial 2 can thus be precisely and/or visually inspected. The tube 20 is removed after the radiopharmaceuticals filled in the vial 2 reaches a predetermined level.

Dosage calibration of the liquid radiopharmaceuticals filled in the vial 2 is then performed. The vial 2 is moved by the manipulating arm 12 from the vial container 21 to a carrier container 24. The carrier container 24 has a T-shaped handle 241, which can be grasped and held by the manipulating arm 12. The carrier container 24 and the vial 2 received in the carrier container 24 are then positioned into a calibrator 3 for measuring the radioactivity of the liquid radiopharmaceuticals filled in the vial 2. Measuring result from the calibrator 3 may then be transmitted to the computer device for processing, which is not related to the present invention and thus will not be further described.

The vial 2, after the calibration operation, is moved, together with the carrier container 24, by the manipulating arm 12 back to the original position where the carrier container 24 was located. The vial 2 is then removed from the carrier container 24 and put back to the vial container 21.

The system further comprises a disposable syringe module 5 comprising a plurality of disposable syringes 51 arranged in a line and a dispensing and injection mechanism 6, both arranged inside the radiation-shielded chamber of the casing 1. The dispensing and injection mechanism 6 is located adjacent both the disposable syringe module 5 and the vial container 21 for accommodating a selected one of the syringes 51 of the disposable syringe module 5. The mechanism 6 moves the selected syringe 51 between a dispensing position where the syringe 51 is located next to the vial container 21 for withdrawing the liquid radiopharmaceuticals contained in the vial 2 into the syringe 51 and an injection position where the radiopharmaceuticals-filled syringe 51 injects the liquid radiopharmaceuticals into a saline water cartridge 70 of a saline water cartridge module 7, which is also arranged inside the radiation-shielded chamber of the casing 1 and will be further described.

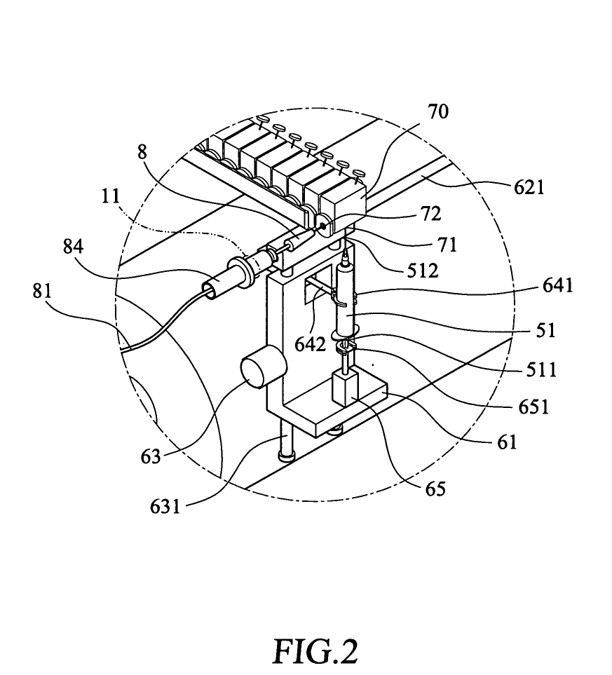

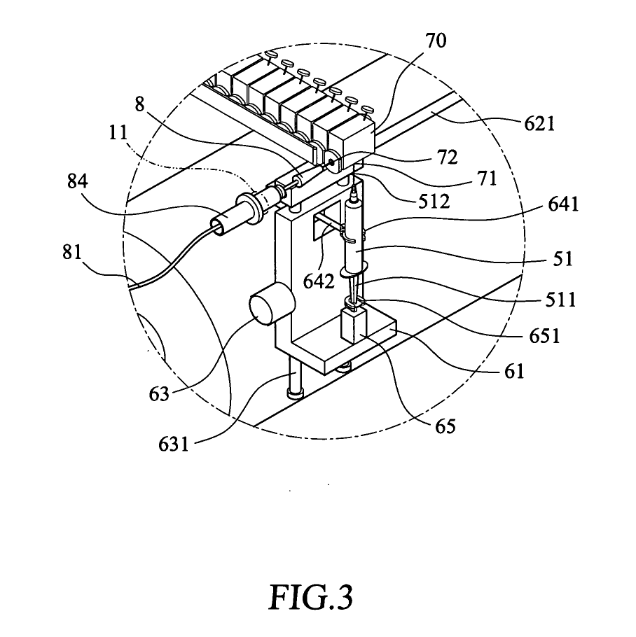

Also referring to FIGS. 2 and 3, both show the selected and radiopharmaceuticals-filled syringe 51 is moved to the injection position by the dispensing and injection mechanism 6 and an injection needle 512 of the syringe 51 penetrating into the cartridge 70 through an block member 71, made of for example rubber or other penetrateable materials, to allow for penetration of the syringe needle 512, but respectively illustrating the conditions before and after the syringe plunger 511 is driven into a barrel of the syringe 51.

The dispensing and injection mechanism 6 comprises a carrier 61, a horizontal transportation device 62, a vertical transportation device 63, a releasable clamping device 64, and a plunger driving device 65. The horizontal transportation device 62 moves the carrier 61 along a horizontal rail 621. The vertical transportation device 63 moves the carrier 61 along a vertical rail 631.

The clamping device 64 comprises a clamp 641, an extendible bar 642, and a controller 643. The extendible bar 642 that supports the clamp 641 is selectively driven by the controller 643 to move the clamp 641 toward the selected and radiopharmaceuticals-filled syringe 51 of the disposable syringe module 5. The clamp 641 then clamps and holds the selected syringe 51.

The plunger driving device 65 comprises a power unit, such as a motor and a power cylinder, for example a pneumatic cylinder, mounted on the carrier 61 at a position corresponding to the plunger 511 of the selected syringe 51 held by the clamp 641. The plunger 511 has a free end configured to releasably engage an end of the plunger 511 and functions to selectively drive the plunger 511 in opposite directions for injection and/or withdrawal of liquid into and/or out of the saline cartridge 70. The plunger driving device 65 comprises a pressure sensor 651 for monitoring the pressure that is applied by the plunger driving device 65 to the plunger 51 to ensure safe injection and withdrawal of liquid into/out of the cartridge 70.

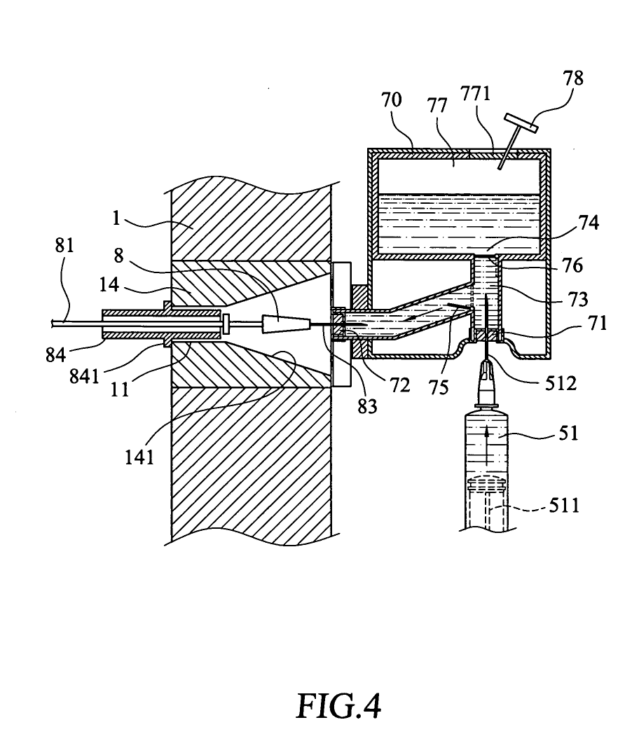

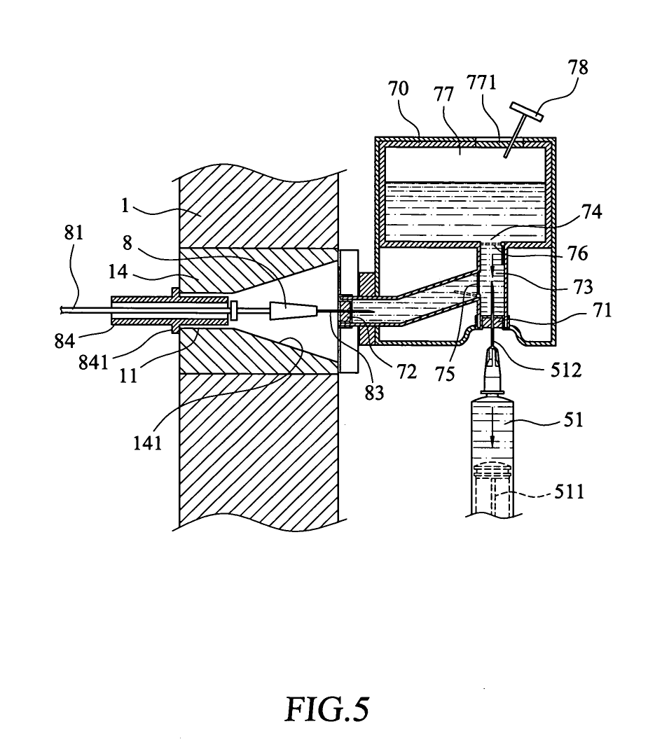

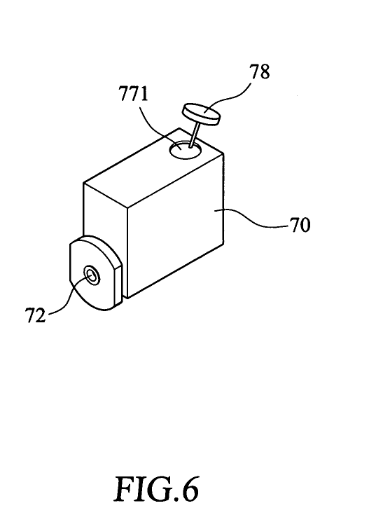

Referring to FIGS. 4 and 5, both show cross-sectional views of the syringe needle 512 of the selected syringe 51 penetrating into the saline water cartridge 70 but illustrating the conditions when the radiopharmaceuticals filled in the syringe barrel is injected into the cartridge 70 and when the saline water of the cartridge 70 is withdrawn into the syringe barrel for flushing of the radiopharmaceuticals, respectively. Also referring to FIG. 6, which shows a perspective view of the cartridge 70, the cartridge 70 comprises a casing defining an interior space forming a saline water reservoir 77 containing a predetermined amount of saline water. The casing of the cartridge 70 forms a radiopharmaceuticals inlet end 71, a radiopharmaceuticals outlet end 72, an internal passage 73, and a saline water outlet 74. The saline water outlet 74 forms an opening for the saline water reservoir 77 and is connected to the internal passage 73 by a one-way membrane valve 76, which allows the saline water to flow out of the saline water reservoir 77 into the internal passage 73, while blocking a reverse flow of the saline water, which may contains undesired impurity, back into the saline water reservoir 77.

The internal passage 73 extends from the outlet 74 to an opening defined in the casing of the cartridge 70, which opening is blocked by a rubber member 71 or other penetrateable member, which functions as the radiopharmaceuticals inlet end 71 of the cartridge 70 through which the syringe needle 512 may penetrate. A branch passage (not labeled) extends from an opening defined in the internal passage 73 midway between the outlet 76 and the radiopharmaceuticals inlet end 71, which opening is closed by a one-way membrane valve 75, to an opening of the casing of the cartridge 70, which is blocked by a rubber blocking member or other penetrateable member 72, serving as the radiopharmaceuticals outlet end of the cartridge 70. The membrane valve 75 allows liquid mixture of saline water and radiopharmaceuticals injected into the internal passage 73 from the syringe 51 to flow from the internal passage 73 to the branch passage, while preventing reverse flow of liquid mixture from the branch passage back into the internal passage 73. Thus, saline water contained in the saline water reservoir 77 is only allowed to flow from the saline water reservoir 77, through the outlet membrane valve 76, the internal passage 73, the membrane valve 75, and the branch passage to the radiopharmaceuticals outlet end of the cartridge 70, as indicated by arrows in FIGS. 4 and 5.

The saline water reservoir 77 of the cartridge 70 forms an opening (not labeled) that is closed by a penetrateable film 771. Saline water can be pre-filled into the reservoir 77 of the cartridge 70 through the opening before the opening is closed by the film 771, or alternatively, saline water can be replenished into the reservoir 77 through injection by an injection means (not shown) penetrating through the film 771 after the opening is closed by the film 771. The film 771 also serves to receive air filter means 78 penetrating through the film 771 to guide atmosphere into the reservoir 77 for pressure balance when the saline water is discharged out of the reservoir 77. The air filter means 78 also functions to remove undesired objects out of the air flowing into the reservoir 77.

An injection device 8 comprises a needle 83, which penetrates through the block member of the radiopharmaceuticals outlet end 72 of the cartridge 70 to guide the liquid radiopharmaceuticals into the injection device 8. A tube 81 extends from the needle 83 through the hole 11 defined in the casing 1 of the system to be connected to a needle 82 (FIG. 1) that is inserted into the body of a patient (not shown). A three-way valve 811, which is manually operable, is provided, comprising a first passage connected to the tube 81, a second passage connected through a filter 812 to the needle 82, and a third passage into which a syringe barrel 813 full of saline water is inserted. The syringe barrel 813 functions to inject saline water into the tube 81, under the control of the valve 811, to expel air inside the tube 81 before the needle 83 penetrates into the radiopharmaceuticals outlet end 72 of the cartridge 70.

The injection device 8 comprises a positioning tube 84 fitting over the tube 81, which supports and helps positioning the needle 83 for penetration through the radiopharmaceuticals outlet end 72. The positioning tube 84 forms a circumferential flange 841, which abuts against an outside surface of the cartridge 70 around the hole 11 to position the needle 83 at predetermined location with respect to the cartridge 70 and to prevent the positioning tube 84 from unexpectedly dropping into the interior space of the cartridge 70.

To provide radiation shielding at the hole 11, an insert 14 made of for example tungsten is inserted into and blocking the hole 11. An aperture (not labeled) sized to snugly receive the positioning tube 84 is defined in the insert 14. The aperture has a diverging, conic inner opening delimited by inclined circumferential surface 141.

In operation, the dispensing and injection mechanism 6 moves the carrier 61, which carries the selected syringe 51, along the horizontal rail 621 to the injection position by the horizontal transportation device 62 and then the vertical transportation device 63 moves the carrier 61 along the vertical rail 631 upward, with which the needle 512 of the syringe 51 is caused to penetrate through the radiopharmaceuticals inlet end 71 of the cartridge 70. Thereafter, the plunger driving device 65 drives the plunger 511 of the syringe 51 to force the radiopharmaceuticals contained in the syringe 51 into the internal passage 73 of the cartridge 73 through the radiopharmaceuticals inlet end 71. Under this situation, as shown in FIG. 4, the membrane valve 76 is closed to prevent the radiopharmaceuticals that is mixed with the saline water inside the internal passage 73 from flowing into the saline water reservoir 77, while the membrane valve 75 is open to allow the radiopharmaceuticals, together with a portion of the saline water container in the internal passage 73, to flow thought the branch passage toward the radiopharmaceuticals outlet end 72 and eventually flowing through the injection device 8 inserted into the radiopharmaceuticals outlet end 72.

When the injection of the radiopharmaceuticals out of the syringe 51 is completed, the plunger 511 is moved downward by the plunger driving device 65 to withdraw a portion of the saline water contained in the internal passage 73 into the syringe 51 for flushing radiopharmaceuticals residual inside the syringe barrel. Under this condition, as shown in FIG. 5, the membrane valve 76 is open to replenish saline water into the internal passage 73, and the membrane valve 75 is closed to prevent the medicine liquid that has been previously driven into the branch passage and the injection device 8 by the injection operation of the syringe 51 from flowing backward. The saline water that is withdrawn into the syringe barrel is then injected into the internal passage 73 and the branch passage again to convey the radiopharmaceuticals residual in the syringe barrel into the internal passage 73. The flushing process can be repeated, if desired.

Although the present invention has been described with reference to the preferred embodiment thereof, it is apparent to those skilled in the art that a variety of modifications and changes may be made without departing from the scope of the present invention which is intended to be defined by the appended claims.

1. A saline water cartridge for radiopharmaceuticals injection, comprising:
a casing defining an interior space forming saline water reservoir in which a predetermined amount of saline water is contained, the casing forming an inlet port and an outlet port sealed by needle penetrateable members, the inlet port adapted to receive a needle of a radiopharmaceuticals-carrying syringe that penetrates through the penetrateable member of the inlet port, the outlet port adapted to receive a needle of a patient injection device that penetrates through the penetrateable member of the outlet port, the reservoir forming an outlet;
primary and secondary passages arranged inside the casing, the primary passage extending from the reservoir outlet, through which the saline water is replenished into the primary passage, to the inlet port of the casing, which allows injection of the radiopharmaceuticals contained in the syringe into the primary passage, the primary passage forming an opening communicating the secondary passage that connects to the outlet port of the casing;
a first one-way membrane vale arranged in the opening of the primary passage to allow the saline water inside the primary passage with which the radiopharmaceuticals injected into the primary passage is mixed to flow into the secondary passage while preventing reverse flow of the mixture of the saline water and the radiopharmaceuticals;
a second one-way membrane valve arranged in the outlet of the saline water reservoir to allow the saline water to flow from the reservoir into the primary passage, while preventing reverse flow of the saline water;
wherein the radiopharmaceuticals contained in the syringe, after being injected into the primary passage through the inlet port of the casing, is prevented from flowing into the saline water reservoir by the second membrane valve and is only allowed to flow to the outlet port through the first membrane valve.

2. The saline water cartridge as claimed in claim 1, wherein the first membrane valve is open when the medicine liquid is injected from the radiopharmaceuticals-carrying syringe into the primary passage of the cartridge, while the second membrane valve is closed.

3. The saline water cartridge as claimed in claim 1, wherein when the saline water is replenished into the primary passage, the first membrane valve is closed, while the second membrane valve is open.

4. The saline water cartridge as claimed in claim 1, wherein the saline water reservoir defines a further opening for filling the predetermined amount of saline water into the reservoir, the further opening being closed by a penetrateable film.

5. The saline water cartridge as claimed in claim 4 further comprising an air filter penetrating through the film that closes the further opening of the saline water reservoir.

6. The saline water cartridge as claimed in claim 4, wherein the predetermined amount of saline water is filled into the saline water reservoir before the further opening is closed by the film.

7. The saline water cartridge as claimed in claim 4, wherein the predetermined amount of saline water is filled into the saline water reservoir by an additional device penetrating through the film after the further opening is closed by the film.
